(19) United States
(12) Patent Application Publication
(10) Pub. No.: US 2013/0061798 A1
(43) Pub. Date: Mar. 14, 2013

(54) Discrete Tunable Sensing Elements and Compositions for Measuring And Reporting Status and/or Product Performance

(71) Applicant: Hans O. RIBI, Hillsborough, CA (US)

(72) Inventor: Hans O. RIBI, Hillsborough, CA (US)

(21) Appl. No.: 13/607,287

(22) Filed: Sep. 7, 2012

Related U.S. Application Data
(63) Continuation of application No. 12/093,370, filed on Sep. 15, 2008, now abandoned.
(60) Provisional application No. 60/736,967, filed on Nov. 14, 2005.

(51) Int. Cl. A61K 8/00; G01N 25/00

(52) U.S. Cl. 116/207; 116/200; 116/206; 252/408.1

(57) ABSTRACT

Discrete sensing elements are described that can be integrated into dental hygiene products and can provide rapid and direct feedback to a user as to oral environmental status and as to the performance of the intended oral care product. Selectively tuned elements can both sense and report relevant information including usage time, adequacy of performance during use, training for better usage, and/or the physical or biological status as can be measured by direct oral contact. Sensing and reporting mechanisms can be tuned for increased or decreased sensitivities. Sensing elements can comprise one or more discrete optical changes, structural changes, or other physical changes to the dental hygiene product as a means of alerting a user of a stimulus. Use of optical reference colors provides a high level of accuracy and objectivity as to the status of a sensing event. Enabling delivery and application compositions and methods are described.

### CROSS-REFERENCE TO RELATED APPLICATIONS

Pursuant to 35 U.S.C. §119(e), this application claims priority to the filing date of the U.S. Provisional Patent Application Ser. No. 60/736,967 filed Nov. 14, 2005; the disclosure of which is herein incorporated by reference.

### BACKGROUND

Sensing elements find use in a variety of different consumer and other products and are employed for a variety of different purposes. Because of their wide-ranging applicability, there is continued interest in the identification of new sensing elements for consumer and other types of products.

### SUMMARY

Discrete sensing elements are described that can be integrated into products including dental hygiene products and can provide rapid and direct feedback to a user as to oral environmental or other status and as to the performance of the intended oral care or other product. Selectively tuned elements can both sense and report relevant information including usage time, adequacy of performance during use, training for better usage, and/or the physical or biological status as can be measured by direct oral contact or other stimuli. Sensing and reporting mechanisms can be tuned for increased or decreased sensitivities. Sensing elements can comprise one or more discrete optical changes, structural changes, or other physical changes to the dental hygiene or other product as a means of alerting a user of a stimuli. Use of optical reference colors provides a high level of accuracy and objectivity as to the status of a sensing event. Enabling delivery and application compositions and methods are described.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### DETAILED DESCRIPTION

The current invention provides for an enabling means to selectively and specifically locate sensing/reporting elements. The elements can be positioned in a wide range of locations and geometries. Combining the multiple features of specific location, geometry, color selection and contrast, reference colors, and the like provides both the manufacturer and user of the product an enabling means to both produce and use the sensing/reporting mechanisms to the greatest extent and at an exceptionally low and affordable cost.

A wide range of different oral products described herein can benefit from similar location color combination referencing and geometrical means. In each case, it is important to provide an accurate sensing mechanism and a clear visual reporting output. The sensing output can be a one-time event or monitor the progression of an oral care process. Sensing elements described comprise one or more discrete optical changes, structural changes, or other physical changes to the dental hygiene product as a means of alerting a user of a stimulus. Enabling features of sensing elements and compositions described include but are not limited to being discretely located and featured as to provide maximum sensitivity and selectivity, the ability to be tuned for maximum performance, and the ability to be specifically designed for optimal visual clarity, viewing and ease-of-use.

Sensing and reporting elements described here within may also find use on a variety of other consumer, household, commercial, industrial, and technological products and product categories where a discrete sensing element can be conveniently, inexpensively, and precisely placed on the product. Conveniently placing a sensing and reporting element at a strategic location on a product that can benefit both the producer and consumer while minimizing the incremental cost can provide a substantial benefit to both manufactures and consumers alike.

Categories of oral care/hygiene products can benefit from a sensing/reporting elements that are appended to or integrated into the product. By way of example but not limitation various oral care/hygiene include: toothbrushes including standard toothbrushes, electric toothbrushes, high frequency sonic toothbrushes, denture brushes, and various toothbrush alternatives; tooth brushes incorporating rubber or other cleaning elements or plastic filaments; denture brushes; toothpicks; bristled between teeth cleaners; plaque removing dental picks; tooth swabs; finger toothbrushes; dental floss; hand-held dental floss holders for adults; dental wax for braces; hand-held dental floss holders for children; night guards; transparent thermal plastic braces; gum stimulators including plastic, rubber tipped metal handled products; wooden stick gum stimulators; dual purpose toothbrush/gum stimulators; medicinal applicators; dental chewing gum; dental retainers; metal braces; invisible/transparent plastic braces; tooth whitening systems, compositions, and devices; dental implants; dental cleaning instruments and dental picks; water picks; dental gauss; dental applicators; dental mirrors; dental power drills including high speed drills; teeth cleaning agents; self-fitting mouth guard including boil-and-bite mouth guards; fillings and filling material; implanted posts; dental coatings; plaque indicators; professional dental tools; tongue cleaners including tongue scrapers, tongue brushes, and tongue swabs; and the like.

Oral care product packaging can benefit from having new features not previously available to manufactures or consumers. By way of example, packaging can have “Try Me” features where a sensing element composition can be placed on a convenient location on a package containing an oral care product that possesses a sensing/reporting element. Sensing/reporting element composition can be positioned and featured on the package such that a consumer can touch, feel and cause an optical change in the sensing element without opening the product. Placing a sensing/reporting element on a package can serve as a particularly important marketing feature for the oral care product manufacturer to promote the sensing/reporting element as a new feature on the product. Alternatively, a sensing/reporting element on an oral care product package can be used as a temper evidence indicator. If a color has changed or been distorted intentionally or unintentionally prior to purchase, the sensing/reporting element can report that product tampering has occurred. Packaging and product features that promote the replacement of an oral care product are of particular importance in order to promote optimal product performance and oral hygiene.

By way of more specific examples, sensing/reporting elements can be specifically employed in selected dental hygiene products to communicate timing and usage information to the hygiene product user. The table below summarizes certain oral hygiene products that benefit from integrated sensing elements by way of example, not limitation:

| Product | Information sensed/reported | Visual output |
|---|---|---|
| Adult toothbrush | 2 minute brushing duration | Reversible color |
| | | change: color to |
| | | color and color to |
| | | colorless-thermal |
| Child's toothbrush | 1-2 minutes brushing duration | Reversible color |
| | | change: color to |
| | | color and color to |
| | | colorless-thermal |
| Toothbrush wear | 2-3 months normal usage | Irreversible color |
| Indicator | | change: color to |
| | | color-auto |
| | | polymerization |
| Toothbrush wear | 2-3 months normal usage | Irreversible color |
| Indicator | | change: color to |
| | | color-shear and |
| | | mechanical induced |
| Dental floss | 2 minutes flossing time | Irreversible color |
| | | change: color to |
| | | color-frictional |
| Dental floss handles | 2 minutes flossing time | Irreversible color |
| | | change: color to |
| | | color-frictional |
| | | on floss. Reversible |
| | | color to color on |
| | | handle-thermal |
| Gum stimulator | 2 minutes stimulation | Reversible color |
| (reusable) | | change: color to |
| | | change and color to |
| | | colorless-thermal |
| Gum stimulator | 2 minutes stimulation time | Irreversible color |
| (disposable) | | change: color to |
| | | color-frictional |
| Chewing gum | Oral cavity temperature | Reversible or |
| | | irreversible color |
| | | change above |
| | | normal body |
| | | temperature |

Other consumer, household, industrial, commercial, and technological products that could benefit from possessing a sensing/reporting element can include, but are not limited to: toys, games, art and craft products, safety products, warning labels, food products, daily use household products, tools, industrial equipment, health care products, personnel care products, cosmetic products, bio-technology products, computer technology related products, printed products, athletic and sports related products, automotive products, consumable products, product packaging and novel packaging features where hot melt compositions are utilized, and the like.

### Positioning of Sensor Elements on a Toothbrush or Other Oral Care Product

Sensing and reporting elements can be placed and positioned on any of a number of locations on a toothbrush or oral care product. By way of example but not limitation, sensing elements can be located on regions including: bristles, brush head backside, brush head side and tip, brush head bristle plane, neck, stem, handle, bristle holes, attachments to brush, inserts in bristle area, placed using co-molded materials at various locations, internally within the body of an oral care product, as an attachment at a specified location on an oral care product, insertion of the element to a slot or insertion location on the oral car product, as a coating, appended as a portion of the product, as an extension on the product or the like. The sensing element of material can be a part of a rubberized element on the brush such as ancillary brushing components contiguous with the bristles.

Sensing elements can comprise from a large portion to a small discrete portion of an oral care device. The sensing element region can occupy up to the majority of an oral care device to a small discrete region strategically located on the oral care device. Selective and discretely placing an element on the oral care device can markedly enhance the sensing and reporting capability of the sensing element.

The sensing element region can occupy from 90% of an oral care device to a visually acceptably visible percentage as low as 0.05%. Usually, the sensing element region will occupy from 50% to 0.1%. More usually, the sensing element region will occupy from 25% to 0.5%. Typically and practically, the sensing element region will occupy from 5% to 1% of the oral care device.

Discretely placing the sensing element on a selected location provides for maximum potential interaction, contact, and accurate reporting. Areas adjacent or nearby to the sensing element can be selectively colorized to serve as reference colors zones that either match the initial color of the sensing element prior to reporting or the final reporting color that the sensing element converts to after a sensing and reporting event has been accomplished.

Oral care products molded with clear resins have the advantage of good optical clarity. Clear resins can include, but are not limited to: copolyester (COPET, PCTA, PCTG), polycarbonate, styrene acrylonitrile (SAN), glycolised polyester terephthalate (glycolised polyester, PETG), styrene butadiene copolymers (SBC), cellulose acetate propionate (CAP), polyester terephthalate (PET) and the like. Such resins can be molded for optimal clarity or be tinted with various transparent dyes production. More opaque resins include polypropylene, ABS, high impact polystyrene, polyethylene, polypropylene, Nylon and the like. Blends of various resins can also be utilized.

Good optical clarity provides the advantage of being able to see a sensing element through the body of an oral care product. An oral care product can be made with a highly clear and untinted resin, slightly tinted resins, moderately tinted resins, heavily tinted resins and translucent resins. For example, a sensing element on the backside of a toothbrush handle can be seen through the side of the head or even through the bristle region of the toothbrush. Seeing the sensing element through the brush allows a brush user to directly see the sensing element without having to rotate the brush handle. In addition, the sensing element can be seen in virtually any position during a brushing session.

The toothbrush bristle can be further sectioned such that there is an open region between the bristles. This provides for a more adequate viewing port between bristles provided that the sensing element is positioned on the backside of the toothbrush handle and the toothbrush is molded with a clear plastic resin. The bristles can be made tufted in two separate sections with a clear region between each tuft section or a circular patch of tufts can be eliminated in the center of the bristle area. Various configurations of bristles can be utilized that optimize the visual appearance of the sensing element during and between brushing.

### Methods for Applying Sensor Elements

Various sensor application formats applied to or a part of oral hygiene product are provided that utilize the compositions described here within including UV optical coatings, film laminates, heat/pressure laminates, ink coatings, tab inserts, injection molded components, pressure sensitive labels, domed adhesive labels, domed plastic elements, printed components, solvent based dipping solutions, painting, hot molten dipping solutions, co-extruding, dip coating, dyeing, sonically welded components, embossed materials, embedding, hot pressing, co-molded materials, powder coated materials, vapor deposited layers, electro-statically adhered material, glued with solvent based compositions, etched components, and hot melt adhesions, elastic or inelastic bands for around the brush handle and close to the brush head, epoxy glues, epoxies comprising sensing materials, cyano-acrylics, cyano-acrylics comprising sensing agents, UV curing agents, water based coatings, solvent based coatings labels, tape, staples that can be firmly embedded in the brush that possesses a sensing element, magnetic adherence, and the like.

The sensor material can be applied by painting, dip coating, ink jet printing, pad printing, lamination, embossing, hot melt application, liquid injection (hot or cold), liquid application, tumbling, impregnation, pressing, dye sublimation printing, chemical treatment, anodization where the oral care product has an aluminum component to it, laser printing and the like. The coating or application method will depend on the composition comprising the sensor material, the desired production process, cost consideration, ease of implementation, and suitability for the final product.

In consideration of selecting an application approach for sensing/reporting elements, cost-of-goods, ease-of-processing, versatility, range and ease of formulation, availability of in-line processing equipment, range of formats, and related features and attributes should be considered in conjunction with the type of sensing element of interest and the type of oral hygiene product for intended integration with.

Hot melt compositions, hot adhesives, hot glue, and related thermoplastic adhesive resins are of general interest since they are particularly amendable to application to an oral hygiene product during an in-line process. The material and processing costs associated with hot-melt compositions is low with respect to other approaches that require pre-fabrication of the sensing element in a form or format that makes the element satisfactory for application. Hot melt compositions and application processes provide for an in-situ approach for forming the sensing element within a feature or directly onto an oral hygiene device or product. Furthermore, hot melt compositions can be formulated with relative ease and utilize temperature and processing conditions that require less investment in capital equipment compared with other fabrication approaches.

By way of example, hot melt adhesion of sensor elements comprising a thermochromic color change, printed messages on brush head over-coated with UV/thermochromic window, affixed sensor tab/labels on the brush head, and printed messages on the brush over-coated with a UV curable thermochromic window can be utilized. Hot melt adhesives comprising sensing compositions are of particular interest due to the ease of locating the composition at virtually any location on an oral care product. For example, hot melt adhesives can be applied to various locations on a toothbrush including: directly to brush bristles, to the brush head, between bristles that have a separation between the bristles, in bristle cavities, along side the brush head, on the brush tip, on the brush neck, along the brush handle, on secondary molded compositions on the brush and any alternative location on the brush that is addressable by application of the hot melt composition.

Hot melt nozzle tips for application of hot melt compositions can be fine or broadened and provide for accurately positioning the hot melt extrusion at any position on a brush. Hot melt compositions can be applied to: the back of the brush head, the brush tip, the brush side, in or over the bristle holes, on the handle near the bristles, along the bristles, between bristles, directly to the bristles in a desired location, and along or on the handle. The hot melt composition can be applied directly to the molded surface or to an impression molded into the brush surface. Applying the hot melt to an impression can provide for a uniform surface profile without any surface protrusion.

Impressions for accepting a hot melt formulation can be pre-molded into an oral hygiene product. The impression can be a simple circular hole, a disc shaped hole, a groove, a pattern, an oval, the shape of a symbol, lettering, a square shape, an insignia, a logo, the shape of a licensed character, or any of a variety of shapes or renderings that correspond to a design of interest. The impression can be completely filled with a single hot melt composition, or multiple compositions to create color change combinations. The sensitivity of a sensing indicator can be tuned by adjusting the depth or configuration of an impression that a sensing material may be applied into.

The impression can range in depth from being very shallow to a deep well. The impression depth can range from 0.001 inch to 0.25 deep. Usually, the impression depth will range from 0.005 inch to 0.15 inch deep. More usually, the depth will range from 0.01 to 0.1 inch deep. Most often, the depth will range from 0.025 to 0.1 inch deep. The depth will depend on the application of interest. Typically the shallower depths will be used for faster acting and reporting sensors whereas deeper depths will be used for slower acting and reporting sensor elements.

### Indicating Mechanisms that can be Utilized as a Sensing and Reporting Means

Optical indicating means can include color change mechanisms and agents, refractive index change materials and mechanisms, optical sensing mechanisms, capillary fill methods using dyed fluid, optical obscuring mechanisms and the like. Examples include but are not limited to: thermochromic, photochromic, mechanochromic, hydrochromic, chemochromic, biochromic, bioluminescent, glow-in-the dark materials, encapsulating melting waxes that melt to reveal an internalized dye, frictionally sensitive color change materials, dye diffusion, mechanical action that leads to dye dispersion, pH induced color change materials, liquid-solid phase transition materials for changing optical clarity, piezochromic materials, electro-chromic mechanisms, and the like. The optical indicating means can be selected based upon the sensing and reporting property of interest. By way of example, a toothbrush duration indicator can utilize a thermochromic color change mechanism whereby oral contact at body temperature for a specified period of time can cause a visual color change in an indicating element alerting an individual that brushing has been minimally accomplished for a recommended period of time.

Alternative outputs that can lead to visual optical changes include but are not limited to: sheer induced mechanisms, shape changes in metal alloys or engineered plastics, strain gauges, piezo-electric induction, reversible stress induced fracturing, and the like. Micro-optical sensing devices such as those employed for medical device applications can find use as sensing means on oral care products (Metrika Inc. CA). Micro-capillary pumping mechanisms can be employed far use as a sensing and reporting means for oral care products (Caliper Inc., CA).

Structural changes signifying timing can be accomplished using memory alloys. Thermally responsive alloys can be integrated into an oral hygiene product such that thermal contact during usage of the product results in a physically observable or otherwise determinable response. By way of example, a toothbrush can respond to usage by undergoing a shape change during use. The shape change occurs in response and correlation to the amount of time used. The shape change response can be configured in a variety of formats depending on the desired physical change intended.

Optical reporting means can take advantage of structural features of the dental or oral hygiene product. For example, the bristles of a toothbrush can be made of a fiber optic grade plastic. Light can be channeled from a light source within the brush handle directly to the brush bristles. The bristles themselves can serve both as an illumination source and as a receptor source for channeling light back to a detector within the brush. Changes in total internal reflectance of received light can serve as a real-time means for monitoring various activities, processes, and conditions during the brushing process.

Real-time optical feedback can provide a direct means for product configurations that can both report and respond to oral hygiene conditions. For example, optical feedback during brushing can be utilized to signal a response whereby during the brushing process an agent can be released from an element of the brush that is intended to assist the brushing process or otherwise provide a benefit such as increasing the concentration of a tooth whitening agent. In another example, an optical feedback can be utilized to activate an ultraviolet light source for activating peroxide-based materials in a brushing medium to enhance whitening activity.

Visual displays can be utilized as an output or readout means including liquid crystal displays, electro-luminescent displays, electro-chromic displays, light emitting diode displays and other convenient display mechanisms that provide a clear and cost effective visual format.

### Multiple Sequential and Simultaneous Plural Indicia

Multiple sequential or simultaneous sensing and reporting elements can be incorporated into dental hygiene products. Often it may be important for more than one sensing means to be present in a product. For example, a toothbrush could have an indicator that senses and reports that its use has expired and should be discarded (e.g. after 3 months use). In addition, the same brush could possess a second indicator for sensing and reporting brushing duration (e.g. 2 minutes brushing time). Two or more different means of sensing and reporting the same process can be utilized on the same oral care product. By way of example, a light emitting diode can be incorporated into a toothbrush head and obscured by a thermochromic agent. The light can be activated by timing mechanisms. Upon a brushing induced color change in the thermochromic layer, the light emitting diode can be revealed.

Multiple sequential color changes can be utilized to indicate the progression and on-going timing of use of a particular oral care product. For example, 2 or more thermochromic materials can be incorporated into a sensing element. The first thermochromic material can be a red to clear color that changes coloration at 25° C. The second thermochromic material can be a blue to clear color that change coloration at 29° C. At the beginning of use, the combined colors will be purple. Upon oral contact and within 30 seconds or less, the purple color will change to a blue color due to the elimination of the red color. On further use of the oral care product and within 60 seconds, the remaining blue color will turn clear such that no residual color remains.

Various combinations of color change and optical pigments can be used in combination to achieve a series of desired effects during the sensing and reporting process. Thermochromic materials can be used with glow-in-the-dark pigments. Thermochromic and glow-in-the-dark pigments can be combined with fluorescent pigments and the like. Thermochromic materials can be combined with photo-chromic materials. Photo-chromic materials can be combined with glow-in-the-dark pigments. A wide range of optical change permutations can be utilized during the sensing and reporting process. In an alternative format, structural changes can be combined with color or optical changes to create a plurality of outputs that together enhance or reinforce the indication.

### Multiple Dye Layers for Specialized Optical Color Changes

Specialized dye layers can be utilized to create unanticipated color change combinations. For example, an underlying red layer can be over-coated with a thermochromic green to clear layer. As temperature increases and the green layer turns clear, the red layer can be exposed. Simply mixing a green pigment with a red pigment would cause a brown appearance. Keeping the green and red colors independent provide a visual means of alerting a user that the beginning and end of use has been accomplished.

### Sensing and Reporting Output Status Examples

Sensing and reporting elements can be integrated into a range of different oral hygiene products to assist in monitoring performance specifications, functions, intended usage, and oral hygiene status. Sensing elements can be used to report vital relevant parameters regarding physical, physiological, biological, chemical, bacteriological, virologic, function. The reporting means and output can be integrated to match the usage function of the oral hygiene product. By way of example, but not limitation, examples of status that can be reported include: brushing duration time; brushing technique; gum stimulation duration time; gum stimulation technique; flossing duration and/or technique; physiologic status including body temperature, hydration, and dental flora; timing for discarding the oral hygiene product; as an oral care technique use trainer; as a sterilization indicator; as a plaque indication sensor; as a salivary enzyme activity sensor; to indicate the possibility of oral bleeding; to indicate possible gum disease; physiologic salivary pH status; salivary antibody status; general health monitoring status based upon salivary constituents and the like. Concentration means can be incorporated into an oral hygiene product that can serve as collection means and subsequent status reporting means.

### Single-Use and Reusable Sensing Elements

An oral hygiene product can integrate single-use or reusable sensing elements. Single-use sensing elements can be attached to the oral care product and provide a one time sensing and reporting event. The single-use sensing element can be replaced after use with a new element for use during the next occasion that an individual intends to use the product. A supply of single-use sensing elements can be supplied separately from the oral care product and attached to the product prior to use. After use, the sensing element can be disposed of.

Reusable sensing elements will typically be permanently attached to the oral care product. A reusable sensing element can be permanently attached to the oral care product by any of a number of durable means. The reusable sensing element should have the capability of a reversible sensing and reporting mechanism so that the element can be regenerated for the next usage.

### Structures and Impressions

Surface structures, impressions, and emblems can be formed from an applied hot melt. During setting from the molten phase to the solid phase, an amorphous hot melt can be molded using a pre-formed chilling anvil. Immediately after the hot melt is applied and prior to setting, the chilling surface can be impressed into the liquefied medium to form a shape of interest. Logos, insignias, messages, features, and a wide variety of structures can be formed. The process can be performed off-line so that injection molding tool changes will not be required.

High resolution holographic patterns can be impressed, embossed, debossed, pressed, or patterned into the surface of an oral hygiene sensor element. The holographic image can present the opportunity for multiple view points and visual outputs. Holographic imaging can provide higher resolution patterning and patterning outputs that appear three-dimensional.

### Detachable and Permanently Affixed Sensing Elements

The sensing element can be permanently affixed to an oral care product or be detachable such that a new or different sensing element can be attached in the same location. This configuration provides for the possibility that a single oral care product can be utilized with different sensing probes depending on the desired function to be monitored. The oral care product can be developed with an attachment position or port such that a sensing element can be directly attached to the product. The sensing element can be developed with a compatible mating mechanism for easy attachment and de-attachment.

### Hot Melt Resin Formulations

Ethyl vinyl acetate/vinyl acetate co-polymer, polyethylene, polypropylene, admixed polyurethane, pure plastic resins with and without added tackifiers and various combinations have been tested. A range of commercially available compositions, as well as custom engineered resins, show feasibility of co-mixing colorants, acceptable operating temperatures, good adhesion, acceptable setting times, and good durability.

Two approaches were pursued for preparation of indicating hot melt compositions. Commercially available and finished hot melt products were further processed by melting followed by the addition of thermochromic colorants or hot melt formulations and colorants were engineered from selected components. Good colorant acceptance was achieved in each case. The adherent properties of the formulated compositions was maintained and tested on polypropylene as well as co-polyester, polyethylene, and polystyrene toothbrush samples.

All compositions and components will need to be certified for oral contact. Component certification matched with processing acceptability and product stability are key priorities. Several vendors and raw materials suppliers have been contacted regarding regulatory acceptance. Certain tackifying agents, resins such as EVA's and polyethylene and polypropylene, waxes, polyolefin waxes, and colorants under consideration should prove to meet acceptable regulatory requirements. Final formulations, concentrations, and loading per brush will need to be established.

A variety of commercial grade hot melt compositions can be modified with sensing/reporting agents. By way of example, hot melt compositions utilized of ream wrap for sheeter mills, hot melt closures, bonding adhesives, freezer grade adhesives, case/carton adhesives, low memory adhesives, speed case sealer materials, multi-purpose assembly adhesives, labeling adhesives, product assembly adhesives, high contact bond adhesives, plastic grade bonding adhesives, hard to bond surface adhesives, low viscosity adhesives, long working time hot melts, low density adhesives, short working time melts, low coloration adhesives, high clarity adhesives, low clarity adhesives, polyethylene based resins, polypropylene adhesives, ethyl vinyl acetate adhesives, and the like.

Hot melt-based sensing/reporting elements have the advantage of ease-of-placement, extremely low cost of materials and application, and versatility of format. Hot melts can be conveniently applied in small to large dot forms, lines, patterns, sequences, designs, line-art, logo forms, insignias, lettering or the like. Hot melt beads can be applied in a continuous or discontinuous form. Application equipment is readily available and adaptable to a variety of oral care, consumer, household, commercial, and industrial products.

### Wax Concentration Ranges and Viscosity Modification

Hydrocarbon or polyolefin waxes can be added to hot melt compositions to reduce the composition viscosity at elevated temperatures. Waxes can range in carbon numbers from 12 carbons to over 100 carbons per molecule. Usually waxes will range in carbon number from 15 carbons to 80 carbons. More usually, hydrocarbon waxes will range in carbon number from 18 to 40 carbons. The hydrocarbon chain can be straight or branched. The hydrocarbon can be saturated or contain unsaturated groups such as double or triple bonds. Various side-chain substituents can be appended on the hydrocarbon including hydroxyl groups, ether groups, polyether groups, carboxylic acids, amides, esters and the like.

Hydrocarbon waxes can be admixed into the hot melt composition during production. Hydrocarbon waxes can be added at rations from 0.01% to 90%. Usually waxes will be added from 0.1% to 70%. More usually, waxes will be added to reduce viscosity from 1% to 50% and typically from 5% to 25%. The amount and type of wax to be added depends on the desired viscosity at an elevated temperature, the melt flow index of the formulated hot melt composition and the adherence characteristics of the final composition. High wax concentrations tend to reduce the adherence characteristics of a hot melt composition.

Hot melt compositions can be formulated such that their viscosities can be varied form a low level (30-200 centistokes) to a high level (10.000 to 100,000 centistokes). Final hot melt compositions can be adjusted in viscosity by resin selection and the addition of viscosity lower agents such as hydrocarbon waxes. Viscosities can be adjusted from 30 to 1,000,000 centistokes. Usually, viscosities will be adjusted from between 50 to 100,000 centistokes. More usually, viscosities will be adjusted from between 100 to 10,000 centistokes and typically from between 100 to 5,000 centistokes.

### Adhesion Enhancements

Hot melt application adherence is enhanced using several different means alone or in combination. Tackifying agents are typically added to the hot melt composition to improve adherence. For polypropylene surfaces, the hot melt adhesion can be enhanced using formulations containing resins more compatible or adherent to polypropylene (e.g. polypropylene or polyethylene based resins). Adhesion can be enhanced using corona treatment (hand held unit or in-line processing). Corona treatment creates chemically more compatible surface by reducing hydrophobic groups and increasing hydrophilic groups. Adhesion can be further promoted between a polypropylene surface and the hot melt by warming or pre-heating the polypropylene prior to the hot melt application. Surface roughening, texturing, or indentations can be introduced on the brush surface to improve entrapment and bonding of the hot melt composition. Injection molded undercuts can be utilized to facilitate anchoring of a hot melt composition applied to an injection molded or machined indent on an oral care product. Furthermore, increasing the application temperature of a hot melt composition helps improve the adherence characteristics of the hot melt composition. Typically application temperatures of between 200 and 500° F. are utilized. More often, application temperatures between 250 and 450° F. will be utilized. Most often, application temperature between 300 and 400° F. will find use as sufficient application temperatures. Higher temperatures tend to improve adhesion whereas lower temperatures tend to mitigate adhesion. Combinations of the above can be further utilized.

### Rapid Setting

Solid surface chilling can be utilized not only for reducing setting times, but also as a means to mold or shape the hot melt composition. By way of example, the chilling means can be used to flatten a hot melt application bead or dot. Chilling surface can typically be maintained between room temperature and subzero. Condensation on the chilling surface may provide an additional non-stick property for release between the chilled hot melt surface and the chilling surface. Chilling surfaces can include but are not limited to: Teflon coated aluminum, Teflon sheet adhered to aluminum, non-stick silicone rubber sheet adhered to a metal rod, various metals including aluminum steel, stainless steel, brass, bronze, copper, oxidized metal surfaces, ceramic coated surfaces, various powder coated metal surfaces and the like. Contact surfaces can be polished, patterned, smooth, regular or other wise textured surfaces. Hot melt compositions can be rapidly impressed, pressed or flattened with a physical object during cooling or self-leveled at elevated temperatures and cooled with a stream of cold gas.

The degree to which the setting time is reduced will depend on the pressure and temperature that the cold pressed surface is applied. Rapid setting times can range from 0.1 second (instant contact) to several seconds (dwell contact). 45° F. Teflon coated aluminum was used for rapid contact of 0.5 to 1 second. Cold pressed surface can range from temperatures well below room temperature yet below the melting transition of the hot melt composition in the range from −100° F. to 150° F. More often, cold pressing surfaces will range from −50° F. to 100° F. Most often, chilling temperature will range from 0° F. to 50° F. Conveniently, chilling may be accomplished using a solid chilled surface or a stream of chilled gas.

### Tackifying Agents and Concentration Ranges

Tackifiers can include rosin based compositions and/or terpene resin based materials. Tackifiers can be included to improve the adherence characteristics of a hot melt composition to typically low adhering substrates such as polypropylene, polyethylene, and co-polyester. The tackifier can be a pure or hydrocarbon mixed composition. The composition can be naturally derived or chemically synthetic. The tackifier is added to a hot melt composition to selectively or non-selectively improve adherence of a hot melt composition to a surface of interest. Tackifiers can be selected and utilized for various properties including adhesion, compatibility with oral contact, stability, and compatibility. Tackifiers are commercially available (Arizona Chemicals, Inc. AZ).

Tackifying agents can be admixed into the hot melt composition during production. Tackifiers are added to increase adhesion between the hot melt composition and the surface that the composition is intended to be applied to. Tackifying agents can be added at rations from 0.01% to 90%. Usually tackifying agents will be added from 0.1% to 70%. More usually, tackifying agents will be added to increase adherence from 1% to 50% and typically from 5% to 25%. The amount and type of tackifying agents to be added depends on the desired adherence characteristics, compatibility of the tackifying composition and compatible optical clarity.

### Hot Melt Hardening Agents

Hardening agents can be added to a hot melt composition to improve the mechanical stability of an applied sensor element. Hardening agents include, but are not limited to high temperature waxes, polyolefin waxes and the like. The addition of waxes can in addition, decrease and improve the viscosity of the molten state hot melt composition.

It is desirable to ensure that an oral cavity sensing element remain intact and permanently affixed to the oral device administering the sensing element. Soft hot melt compositions may be adversely sheared or partially removed by contact with teeth during use. The addition of hardening waxes can significantly overcome the weaknesses of non-hardened compositions.

Hardening agents can be added in the range of 0.1 to over 80%. Usually, the hardening agent will be added at a concentration of 1% to 50%. More usually, the hardening agent will be added from 5% to 30%. Most often hardening agents will be added in the range of 10% to 25%. The exact concentration will depend on the desired hardness and related properties affected by the addition of the hardening agent.

### Hot Melt Production Forms

Hot melt indicating formulations can be conveniently cast or formed into a variety of shapes and configurations useful for processing, production, and manufacturing. Production and processing equipment often vary in the type, shape, volume, and delivery of a hot melt composition and, therefore, it is important to form or cast an indicating formulation into a relevant and convenient format. Formats for casting or forming production grade lots of indicating hot melt formulations can include, but are not limited to: small pellets (equal to or small than 0.1 inch in diameter), medium size pellets (0.1 inch to 0.25 inches in diameter); large pellets (0.25 inch to 1.0 inches in diameter); small and large discs (0.1 inch to several inches in diameter); cubes; tiles; rods; ingots; slugs; granulated forms; chopped forms; sheets; sticks; blocks; dowels; cord; extruded shapes; tubes; balls; molded shapes; slab forms; various molded pan shapes; various cast shapes; bullion; shapes meant to fit a particular heating pot; and the like.

### Production Examples

In-line hot melt extrusion/applicator can be used to apply a fully formulated hot melt indicating composition. The hot melt composition will be supplied in bulk form and added to a heating tank in the extrusion applicator. The dwell time of the hot melt composition should be minimized to 1 hour or less at elevated temperatures (350° F. or above) in order to preserve the activity of thermochromic agents employed. The bulk hot melt composition can be added in solid form using an auto-feeder. A metered amount of liquefied hot melt composition will be supplied to a heated line and directed to a controlled application nozzle. The nozzle may be pressure actuated for delivery of a metered amount. If incremental adhesion is required, an in-line corona treatment may be required. Corona treatment would be accomplished prior to hot melt application.

A recessed circle or compatible hole-shape will be pre-formed in the brush by injection molding. An insertion pin can be added to an existing or new mold in the desired dimension and position. Otherwise, the cavity can be designed and produced with the ultimate feature of interest. The recessed hole will be used to accept a metered amount of applied hot melt composition. The recess volume and hot melt volume will match so that the final sensor configuration will be level with the brush surface.

Immediately following application of the hot melt composition, the hot melt bead will be pressed into a level film so that the recess hole is completely filled and level. Pressing/chilling times are anticipated to be one second or less. The final hot/melt sensor surface can have a smooth or textured finish depending on specifications. Brushes will be immediately ready for any final manufacture steps required or for final packaging.

Recessed holes or indents may also find use for off-setting an applied label, decal, tab, tile or the like. The recession can serve to maintain a level surface on an oral care device. Smooth surfaces are desirable to ensure comfort during use and to avoid any oral cavity irritation.

Sensing elements can be added or applied to a brush or oral care product at any of a number of introduction points during production. The sensing element can be added immediately during the injection molding phase of a toothbrush or product body. The element can be added directly after molding of the product or brush body, but before feature of the product are added. In case the product is a toothbrush, the sensing element can be applied before or prior to adding bristles to the brush. Good adherence of the sensing element is required if the element is to be added prior to bristling. The bristling process often creates aggressive vibrations and shock. Hot melt applications have the advantage of strong adhesion and provide the advantage of stable adherence of an element during and after bristling.

### General Configurations and Compositions for Adjusting and Controlling Response Time of Indicating Sensor

The response time for an indicator means can be adjusted to be an immediate response or a delayed response. The response time can occur between a millisecond and 24 hours. Usually, the response time will be adjusted between 0.1 seconds and 1 hour. More usually, the response time will be between 1 second and 30 minutes. Typically, the response time will be set for products to be during the prescribed usage time of an oral care product such as a toothbrush between 30 seconds and 3 minutes. Dentists often advise that teeth should be brushed effectively for from one minute to 3 or more minutes. Indicators described herein can be adjusted to provide an accurate visual sensing and reporting process that alerts an individual that they have brushed for a recommended period of time.

Brushing duration timing can be controlled using one or more design elements or parameters including: thermochromic color change temperature transition; thermochromic colorant concentration for complete color clearing; secondary colorant utilized for signaling complete color change conversion; thickness of sensor layer; employing the use of a transparent overlay as a thermal delay; adjusting the thermal conductivity of the resin; depth of embedding the sensor element; position of the sensor element on the brush relative to oral contact during brushing; lighter or darker reference colors and hues for comparison of end point settings; and the like.

Different means can be employed to adjust and tune the desired response time of an indicator means. The response time can be adjusted using a thermal delay means such as a transparent insulating window or insulating layer that covers a sensor or indicator. The window can create a delay response by insulating a temperature, chemical, or other physical activation means. The thicker the window, the longer the delay can be established.

Insulating layers can be solid, liquid or gaseous. The resonance time delay between when a sensor surface is exposed to a change in ambient temperature and when an indicating means is raised to the ambient temperature depends on the thermal characteristics of the separating medium between the surface and the indicating means, time, and thermal contact during usage.

The thermal properties of a sensing element can be coupled to or decoupled from an oral care product. A sensing element can be directly adhered to an oral care product and in direct thermal contact with the oral care product. In this scenario, the oral care product can act as a heat sink to the sensing element and facilitate a delay in the timing of when the sensing element may respond. Alternatively, a sensing element may be separated from direct contact with an oral care product by utilizing an insulating layer between the sensing element and the oral care product. In this scenario, the oral care product's thermal characteristics would not impact the thermal characteristics of the sensing element. In either scenario, the thermal delay characteristics of the sensing element can be designed to utilize or not utilize the thermal characteristics of the oral care product that the element is attached to.

Timing delay windows can be comprised with clear plastic, optical ultraviolet cure resin as an over coat, clear pressure sensitive labels, domed labels made with transparent coatings (stiff or flexible), color change agent homogeneously mixed in thickened transparent or semi-transparent layer, laminates, laminates containing liquid crystal pastes or oils, a cavity filled with a low thermally conductive gas or a vacuum or the like

By way of example, a time delay window can range from a 1 micron film to two centimeters. Usually, the window will have a thickness from 10 microns to 1 centimeter. More usually, the window will have a thickness from 0.1 millimeters to 5 millimeters. Most often a window will have a thickness from 0.2 millimeters to 2 millimeters. Typically a practical thickness will be utilized that will help to create a response within the desired time frame of use for an oral hygiene product.

Sensing/reporting elements can be fully embedded and internalized with in the body of an oral care or other product when transparent or optically clear resins are utilized for making the product. By way of example, a sensing element can be embedded, placed or molded within the interior of a product. The resin utilized in molding the product can then be gated to completely encapsulate and seal the sensing element. The molded layer covering the sensing element can be used to act as a thermal delay window. The thickness of the thermal delay window produced in the molding/encapsulation process can range in thicknesses similar to those described above. Transparent or translucent resins form encapsulating or molding/sealing a sensing/reporting element can include, but are not limited to: copolyester (COPET, PCTA, PCTG), polycarbonate, styrene acrylonitrile (SAN), glycolised polyester terephthalate (glycolised polyester, PETG), styrene butadiene copolymers (SBC), cellulose acetate propionate (CAP), polyester terephthalate (PET) and the like. Such resins can be molded for optimal clarity or be tinted with various transparent dyes production.

Alternatively, a thermally responsive chromic change agent can be evenly or unevenly distributed throughout the layer rather than be coated by an optically clear window. The concentration of the chromic change agent can be adjusted along with the total thickness of the layer such that the optical density of the layer can provide a sufficient initial and final color contrast change so that a visual determination can easily be distinguished during a sensing event.

The opacity or optical clarity of the sensing/reporting element composition can be further utilized to affect the visual timing of the element. Increased opacity in a sensing/reporting element reduces the visualization of deeper regions within the sensing element surface. As the color change is initiated, only the outer layers visually appear to turn color. The lower or more deeply embedded layers are obscured by the outer layers and therefore are not seen to change or not change color. Increased optical clarity in a sensing/reporting element permits light to more deeply penetrate the sensing layer. The lower or more deeply embedded layers are more apparent than with opaque sensing elements. The perceived time delay in sensing/reporting elements that identical sensing formulations can be increased using more transparent or optically clear mediums. The perceived time delay in a sensing/reporting element can be reduced using opacifying components mixed into the medium. The degree to which it is judged what time delay should be utilized can be adjusted by changing the concentration of an added opacifying agent.

Opacifying agents can include dyes, titanium dioxide, micro-particulates, talc, microcrystalline materials, optically dense additives, various fine powders, calcium carbonate, micro-balloons, nano-particles, non-mixing polymers, opacifing polymers and the like. Opacifying agents can be added from over 90% by weight to a sensing/reporting formulations to as low as 0.01%. More usually, the opacifying agent will be added from 70% to 0.1%. Typically, the opacifying agent will be added from 50% to 1%. Most often, the agent will be added from 25% to 5% by weight.

Higher optical clarity in an optical sensing dye can be achieved by utilizing smaller grain sizes of the optical agent. Grain sizes can be produced to the submicron level or below. Smaller grain sizes of chromic change agents permits more light to travel through the medium and medium comprising the sensing/reporting element. Grain sized for the optical change agent can be from 100 microns or greater to molecular sizes of 10 Angstroms. More often, the dye grain size will be from 50 microns to 10 nanometers. More usually, the dye particle size will be from 10 microns to 100 nanometers. Most often the grain size for more transparent compositions will be practically from 500 nanometers to 1 micron in diameter.

As an alternative thermal insulating layers or windows delay and timing mechanisms can be accomplished using other means such as chromatographic migration layers, capillary flow layers, dissolving mediums, mixing mediums, slow release layers, mechanical sheer and removal surfaces, chemical change mechanisms, and the like.

More than physical or chemical characteristics can be used in combination for fine-tuning the duration response time of a sensing/reporting element. For example, very short response times can be achieved using a low temperature thermochromic agent at low concentrations in a thin layer without a thermal delay window. Very long response times can be achieved using a high temperature thermochromic agent at high concentrations in a thick layer and with a thermal delay window. Permutations of various properties can be used to achieve a medium time delay. It will be practical to utilize certain chemical or physical properties for certain products. The exact combination of sensing/reporting element properties will depend on the product of interest, manufacturing process intended, cost-of-goods, features desired and the like.

### Film-Based Sensing Indicators than can be Adhered to an Oral Hygiene Product

Sensing and reporting elements can be pre-fabricated utilizing plastic compositions that comprise both a sensing reporting optical dye or pigment and a plastic composition that utilizes any of a variety of standard and engineered resins. The sensing/reporting optical dye or pigment can be added during the extrusion process, during film or sheet formation, painted, printed, or otherwise applied to the plastic composition.

The film-based sensing/reporting elements can be made with a wide range of convenient thermal plastics. The element can be pressure molded, injection molded, thermoformed or vacuum formed, or the like. Plastics and polymer compositions used for making the tray include: polyvinyl chloride (PVC), various polyolefins such as polypropylene and polyethylene, high density polyethylene (HDPE), low density polyethylene (LDPE), cross-linked high-density polyethylene (XLPE), softened acrylic, ABS, thick Kapton™ tape materials, Teflon® (polytetrafluoroethylene (PTFE), tetrafluoroethylene TFE and fluorinated ethylene polypropylene FEP)-based materials, brand names such as Kydex, polystyrene, thermoplastic polyesters, nylon, styrene-butadiene, epoxy casts, polybutylene, TPX (poly(methyl pentene), terephtalate polyethylene (PET), PETE, PETF, polyethylene teraphthalate G copolymer (PETG), polysulfone (PSF), polyurethane (PUR) Thermanox™ (TMX), polymethylmethacrylate, and the like. Strong flexible plastics such as polycarbonate are often desirable. Polycarbonate can be thermoformed, pressure formed, and injection molded.

Other exemplary plastics may include, but are not limited to: ethylenechlorotrifluoreethylene (ECTFE), ethylentetrafluorethylene (ETFE), polinvinylidene fluoride (PVDF), ethylene-propylene rubber (EPR), silicone rubber (SI), Alcryn® thermoplastic rubber (TPR), HT thermoplastic rubber (HTPR), Santoprene® thermoplastic rubber (TPR), LSOH cross-linked compounds, LSOH thermoplastic compounds, methylvinyletherfluoralkoxy (MFA), perflouroalkoxy (PFA), thermoplastic polyester elastomer (TPE), polyimide (Kapton®), polyurethane (PUR), polyvinyl chloride 105° C. (PVC), polyvinyl chloride 70° C. (PVC), low temperature polyvinyl chloride (LTPVC), oil resistant Polyvinyl chloride (OR PVC), semi rigid polyvinyl (SR PVC), polyvinyl chloride polyurethane (PVC PUR), copolyester (COPET, PCTA, PCTG), polycarbonate, styrene acrylonitrile (SAN), glycolised polyester terephthalate (glycolised polyester, PETG), styrene Butadiene copolymers (SBC), cellulose acetate propionate (CAP), and the like.

### Sensing Progression/Timing Features

Differential, metered, sequentially timed, and timing progression can be accomplished by using various thicknesses or depths of a sensing composition. By way of example, hot melt compositions, injection molded sensing elements, or otherwise attached sensing elements can have a depth gradient running along the length of a sensing element. The thinnest portion of the sensing element can be formulated to respond immediately to an oral care process. Progression of the response can be made to continue along the length of a sensing element such that the thickest portion will respond last to the oral care process.

Time sequential indicator designs can include elongated channels that are filled with a sensing composition where the channel is shallowest at one end and deepest at the other end. Circular wells can be made where the depth of the well is shallowest at the center of the well and deepest at the edges of the well to be filled with a sensing composition. Circular wells can alternatively be deepest at the center and shallowest at the edge. Alternatively, a pedestal or structural feature can be present at the center of a well geometry such that the feature remains lower than the top edge of the well, but would still be submersed beneath the sensing composition intended to fill the well. The pedestal or feature would provide a rapid indication to a sensing event where the surrounding deeper portions of the well would provide a more delayed response. Bulls-eye targets or other features can be utilized to provide an understandable and recognizable color change response to an oral care indication. Spiraled patterns and inclined features can be utilized within a well such that a timing clock-like feature can be utilized to display the progression of an oral care indication.

Sequenced timing events can also be pre-determined using discrete formulations of sensing compositions. The individual formulations can be prepared to react to an oral care stimuli at pre-selected times. The discrete formulations can be applied to an oral care product adjacent to one another such that each sensing element comprising the discrete formulation can change color in a sequential process. By way of example, toothbrush timing indicators can be formulated for two or more times common to brushing. The first zone can indicate that 30 seconds have passed. A second zone can indicate that 60 seconds have passed. A third zone can indicate that 90 seconds have passed. A fourth zone can indicate the 120 seconds have passed and that brushing is complete. The zones can comprise the same color change type or different colors to indicate different timing events.

Sensing/reporting elements can be made with either abrupt color change transitions to indicate a “not done” to “done” transition within a narrow time frame or can be made with a gradual color change to indicate that a sensing event is in progress and that the activity should be continued until the color change is complete. The use of an abrupt or gradual color change will depend on the product configuration desired. Certain products will find advantage with an abrupt change so that there is no ambiguity regarding completion time. Other products will find advantage using a gradual change so as to keep the user alert and can anticipate when the process will be complete.

Key thermal transition variables for controlling brushing duration can include but are not limited to temperatures of 22° C., 23° C., 24° C., 25° C., 26° C., 27° C., 28° C., 29° C., 30° C., 31° C., 32° C., 33° C., 34° C., 35° C., 36° C., and 37° C. To date, the best results for 30 second to 2 minute brushing times were obtained using 27° C., 31° C. and 35° C. Shorter delay times of 30-60 seconds can be achieved with 27° C., moderate delay times of 60-120 seconds were achieved using 31° C. Rapid color change times were seen at 25° C. or lower. Extended or indistinguishable changes were seen above 35° C.

Oral cavity temperatures and conductive temperatures to an oral care device must be considered when employing an appropriate temperature transition for a sensing/reporting element. Bodily temperatures, flushing the mouth with a particular water temperature, the style of usage of an oral care product, the direct or indirect contact time between the oral cavity and the oral care product, evaporative cooling of water from the oral care product during cooling, the degree of agitation of the oral care product with the mouth during usage and the like should be considered during design and selection of the physical and chemical characteristics of the sensing/reporting element to be produced.

### Liquid Crystal Indicators

Liquid crystal gel laminate with pearlescence and/or iridescent color change effects can be utilized as a sensing and indicting means on oral hygiene products. Liquid crystal strips can register one or more color change effects indicating a single duration time or dual or multiple sequential timing durations. Liquid crystals can be printed on a laminating layer or can be in a liquefied or gel form and encased or laminated between two layers to create a thick fluid layer.

One or more liquid crystal indicating zone may be present. Multiple indicating zones can be utilized to monitor or observe a particular oral stimuli. By way of example, three temperature levels can be placed side-by-side on a toothbrush. The first zone can be set at a low temperature (e.g. 25 degrees C.), the second zone at a moderate temperature (e.g. 27 degrees C.) and the third zone set at a higher temperature (e.g. 29 degrees C.). During use, the first zone can indicate the initial use of an oral device has been initiated. The second zone can indicate that use is near completion, and the third zone can be used to indicate that use has been successfully completed.

Protective coatings can be used to cover a liquid crystal layer. The protective coating can be a polyester film or any of a variety of clear plastic films. The plastic film can serve both to protect the liquid crystal layer as well as a thermal delay layer. Thicker films can be used to further delay a thermal response in the liquid crystal layer. Thinner layers can be used to keep the response time to a minimum.

### Optical Reporting Dyes and Pigments

Thermochromic dyes can find use in a variety of oral care or oral hygiene applications and formats. Thermochromic dyes can include but are not limited to compounds including: bis(2-amino-4-oxo-6-methylpyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the Photoproduct 7,7adihydro-4,7,7,7a-tetramethyl-2-phenylbenzo[b]thiophene-5,6-dicarboxylic anhydride, and the like.

Other thermochromic dyes of interest include leucodyes including color to colorless and color to color formulations, vinylphenylmethane-leucocynides and derivatives, fluoran dyes and derivatives, thermochromic pigments, micro and nano-pigments, molybdenum compounds, doped or undoped vanadium dioxide, indolinospirochromenes, melting waxes, encapsulated dyes, liquid crystalline materials, cholesteric liquid crystalline materials, spiropyrans, polybithiophenes, bipyridine materials, microencapsulated, mercury chloride dyes, tin complexes, combination thermochromic/photochromic materials, heat formable materials which change structure based on temperature, natural thermochromic materials such as pigments in beans, various thermochromic inks sold by Securink Corp. (Springfield, Va.), Matsui Corp., Liquid Crystal Research Crop., or any acceptable thermochromic materials with the capacity to report a temperature change or can be photo-stimulated and the like. The chromic change agent selected will depend on a number of factors including cost, material loading, color change desired, levels or color hue change, reversibility or irreversibility, stability, and the like.

Alternative thermochromic materials can be utilized including, but not limited to: light-induced metastable state in a thermochromic copper (II) complex Chem. Commun., 2002, (15), 1578-1579 under goes a color change from red to purple for a thermochromic complex, [Cu(dieten)2](BF4)2 (dieter=N,N-diethylethylenediamine); encapsulated pigmented materials from Omega Engineering Inc.; bis(2-amino-4-oxo-6-methyl-pyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the Photoproduct 7,7adihydro-4,7,7,7a-tetramethyl-2-phenylbenzo[b]thiophene-5,6-dicarboxylic anhydride, and the like. Encapsulated leuco dyes are of interest since they can be easily processed in a variety of formats into a plastic or putty matrix. Liquid crystal materials can be conveniently applied as paints or inks to surfaces of color/shape/memory composites.

Photochromic dyes can find use in a variety of color change mediums and formats. Photochromic materials can include but are not limited to dyes including: 1,3-Dihydro-1,3,3-trimethylspiro[2H-indole-2,3′-[3H]phenanthr[9,10-b](1,4)oxazine]; bicyclo[2.2.1]hepta-2,5-diene; benzyl viologen dichloride; 4,4′-bipyridyl; 6-bromo-1′,3′-dihydro-1′,3′,3′-trimethyl-8-nitrospiro[2H; 5-chloro-1,3-dihydro-1,3,3-trimethylspiro[2H-indole-2,3′-(3H)naphth[2,1-b](1,4)oxazine]; 6,8-dibromo-1′,3′-dihydro-1′,3′,3′-trimethylspiro[2H; 1,1′-diheptyl-4,4′-bipyridinium dibromide; 1′,3′-dihydro-5′-methoxy-1′,3′,3′; 1′,3′-dihydro-8-methoxy-1′,3′,3′-trimethyl-6-nitrospiro[2H]; 1′,3′-dihydro-1′3′,3′-trimethyl-6-nitrospiro[2H-1-benzopyran-2,2′-(2H)-indole]; 1,3-dihydro-1,3,3-trimethylspiro [2H-Indole-2,3′-[3H]naphth[2,1-b] [1,4]oxazine]; 1,1′-dimethyl-4,4′-bipyridinium dichloride; 5-chloro-1,3-Dihydro-1,3,3-trimethylspiro[2H-indole-2,3′-(3H)phenanthr[9,10-b](1,4)oxazine]; 5-methoxy-1,3,3-trimethylspiro[indoline-2,3′-[3H]naphtho[2,1-b]pyran]; 2,3,3-trimethyl-1-propyl-3H-indolium iodide and the like.

Thermochromic color to colorless options can include by way of example, but not by limitation: yellow to colorless, orange to color less, red to colorless, pink to colorless, magenta to colorless, purple to colorless, blue to colorless, turquoise to colorless, green to colorless, brown to colorless, black to colorless. Both hued to hueless color state changes and hueless to hued color state changes can be employed. Color state changes can include gradual changes from one color to another, rapid color changes from one color to another, and color state changes that have a transition history (e.g. the reversal of the color change can be delayed by formulating the composition to slowly reverse in color change). Color change histories have the advantage of prolonged color indication that a oral care process has properly taken place and can be viewed by other individuals within a specified time interval.

Color to color options include but are not limited to: orange to yellow, orange to pink, orange to very light green, orange to peach; red to yellow, red to orange, red to pink, red to light green, red to peach; magenta to yellow, magenta to orange, magenta to pink, magenta to light green, magenta to light blue; purple to red, purple to pink, purple to blue; blue to pink; blue to light green, dark blue to light yellow, dark blue to light green, dark blue to light blue; turquoise to light green, turquoise to light blue, turquoise to light yellow, turquoise to light peach, turquoise to light pink; green to yellow, dark green to orange, dark green to light green, dark green to light pink; brown and black to a variety of assorted colors, and the like. Colors can be deeply enriched using fluorescent and glow-in-the-dark or photo-luminescent pigments as well as related color additives.

Photo-luminescent compounds can find use in a variety of color change mediums and formats. Photo-luminescent compounds can include, but are not limited to, a variety of materials. Greens, green blue and violet can be made with alkaline earth aluminates activated by rare earth ions. By way of example, strontium aluminate can be activated using europium (SrAl03:Eu). Visual wavelengths can include: green at 520 nm, blue-green at 505 nm, and blue at 490 nm. Red and orange colors can be generated with zinc sulfide.

Fluorescent dyes can find use as additive colorants or background colorants to various color to colorless thermochromic dyes. Fluorescent dye compounds can include but are not limited to: fluorescein, fluoresceine, resourcinolphthalein, rhodamine, imidazolium cations, pyridoimidazolium cations, dinitrophenyl, tetramethylrhodamine and the like. A wide range of fluorescent dyes that can be activated at various wavelengths and emit light at lower wavelengths can be purchase from Sigma-Aldrich (Saint Louis Mo.) or Molecular Probes (Eugene Oreg.). Fluorescent dye pigments are commercially available form various vendors (e.g. Day-Glo Corporation, Cleveland, Ohio)

Other optical pigments can be added to the thermal impression setting composition to create a variety of practical optical effects. By way of example, glow-in-the dark pigments may be added in order to help an individual locate the oral care product in a dark room. Photo-luminescent compounds can find use in a variety of color change oral care product mediums and formats. Photo-luminescent compounds can include but are not limited to a variety of materials. Greens, green blue and violet can be made with alkaline earth aluminates activated by rare earth ions. By way of example, strontium aluminate can be activated using europium (SrAl03:Eu). Visual wavelengths can include: green at 520 nm, blue-green at 505 nm, and blue at 490 nm. Green, red and orange colors can be generated with are zinc sulfide. Color change and/or luminescent materials can include but are not limited to photo-luminescent material such as glow-in-the dark complexes such as copper doped zinc sulfide (Hanovia Corporation).

Of particular interest are classes of polymeric thermochromic dyes such as polydiacetylenic compounds, polythiophenes, and other polymeric materials that possess thermochromic color change characterizes. Polymer based thermochromic materials like polydiacetylenes have certain advantages including their large molecular weight and adjustable physical characteristics. Polydiacetylenes also possess the optical thermochromic property of undergoing a continuous hue change during a thermochromic transition. Initially at low temperatures, they can exist in a deep blue state. As the temperature is increased they begin to change their conjugation length and blue hues become mixed with red hues to appear deep purple. As the temperature continues to increase toward the transition temperature, the red hue dominate and magenta colors begin to appear. At temperature close to the transition temperature, red hue over come any residual blue tones and the color becomes deep red. At the transition temperature, the polymer exhibits primarily a red hue. As the temperature rises above the transition, the color becomes orange. At even higher temperatures above the transition temperature, the color becomes bright yellow orange.

Optical reporting pigments of dyes can serve as indication means to help alert an individual of proper handling condition during use. Thermochromic dyes or pigments can be used as an indicating means to visually determine when the impression compositing has achieved the correct softening level during initial heating. The thermochromic change can indicate the ideal impression formation temperature. The thermochromic color change can find use to indicate an adequate temperature for re-molding the impression material for devices intended to be reused and refitted. A thermochromic dye or pigment can importantly indicate a critically high temperature that could cause scalding and oral burns during initial fitting. A thermochromic change can provide a benefit by indicating when a device has been adequately sterilized prior to initial or subsequent use. A thermochromic dye or pigment can be used to indicate when an ideal temperature has been achieved to release an active component for time impression material matrix such as a whitening agent or medicinal compound. The thermochromic change can be a single distinct visual change. More than one thermochromic change can be used to indicate more than one temperature criteria. The thermochromic change can be distinct from one color to another, from a colorless state to a colored state, from a colored state to a colorless state, undergo a continuous hue change throughout a complete thermal cycling of an impression device, or the like.

The thermochromic change can be irreversible or reversible. An irreversible thermochromic change can be used as a single event to indicate that a single temperature objective has been met. By way of example, it may be desirable that the color changes from one color to another to indicate that the device is specifically ready for insertion after heating has been accomplished or that the device has been sterilized a single time and is safe to use.

Reversible color changes can find a wide variety of uses in that that the color change can be recycled and repeated during repeat uses of the device can successfully report multiple sequential temperature setting before, during and after the impression setting process. More often reversible color changes can be utilized repeatedly for multiple indication means and provided continual value since if device is intended for re-fitting, then the thermochromic change should be reversible.

A dye system can be used as a wear-use indication means that reports that the device has been adequately used and should be discard and replaced with another new device of the same type. The substrate material and indicating dye can be adjusted jointly to ensure that when continued use begins to exhibit a discrete color change or the like signaling that device should be disposed of and not to be continued to use.

### Thermochromic Colorant Concentration

Thermochromic agents can be utilized as an indicating means by adding the agent in an appropriate form depending on the composition of the indicating formulation. A thermochromic agent can be added from concentrations as low as 0.01% to over 90%. Usually a thermochromic agent will be added at concentrations ranging from 0.1% to 70%. More usually, thermochromic agents will be added from between 1% and 25% and typically from between 2% and 15%. The concentration of thermochromic agent utilized will depend on the specific application of interest, the desired color contrast to be achieved, the richness of coloration achieved, the time delay of interest desired, and consideration of material cost and practicality.

### Active and Inactive Dye/Colorant Additive Concentrations

Various inactive and active dyes can be added at concentrations of interest to achieve an optical effect of interest. The dye can be inactive such as an FD & C dye or active such as a glow-in-the-dark pigment or fluorescent dye. Dyes can be added from concentrations as low as 0.001% to over 90%. Usually a dye will be added at concentrations ranging from 0.01% to 50%. More usually, thermochromic agents will be added from between 0.1% and 25% and more often from between 0.5% and 15%. Most often concentrations between 1% and 5% will find use in most applications. The concentration of dye utilized will depend on the specific application of interest, the desired color contrast to be achieved, the richness of coloration achieved, and consideration of material cost and practicality.

### Contrast Matching with Reference Colors on Brush

Various regions, sections or elements of an oral care product can be comprised with an inactive color zone or have a feature on the product that possess a particular coloration. The inactive or stationary color can be used as a reference color to match either an initial color or a secondary (changed color) and serve as a reference color for comparison. By way of example, the handle of a toothbrush may have one of its compositions or features colorized with a particular hue as a reference color. Likewise, the initial color of a sensing element may be colorized with a color change composition that matches the composition of feature on the toothbrush handle. During a sensing and reporting event, the initial color of the sensing element will change such that the color match between the reference color and the sensing element will be contrasted.

Similarly, the reference color may be colorized to match a secondary color that the sensing element may change to. By way of example, a toothbrush may have a feature that has a particular hue or color that is initially different than the starting color of a sensing element. During a sensing and reporting event, the color of the sensing element will change from a contrasting color with respect to the reference color to a color that matches the reference color.

Unlike descriptions in U.S. Pat. No. 4,957,949, it is important that a discrete and readable sensing element be positioned such that a clear and unambiguous reporting response can be achieved. U.S. Pat. No. 4,957,949 describes a method by which that the entire toothbrush changes color either on the handle or the toothbrush head. Describe sensing/reporting elements can be placed in specific locations such as the back of a toothbrush head. Importantly, adjacent to the sensing element can be a reference color zone that indicates and matches a color that the reporting sensing element either starts with or ends with. Alternatively, discrete sensing element when used can changed from a particular color utilized in the toothbrush itself to another color. For example, the sensing reporting element can be placed in a small diameter circular pattern on the back of the toothbrush head. The sensing/reporting element can have an initial color that is significantly different in contrast from the toothbrush body. As brushing occurs and the brushing duration limit has been achieved, the sensing/reporting element can become the same color as the toothbrush body and therefore indicate precisely that the adequate brushing duration has been achieved.

Assuming the toothbrush body is a non-colored white composite, the sensing/reporting element can be any of a wide range of initial starting colors. Colors can include but are not limited: red, blue, green, yellow, magenta, pink, purple, turquoise, orange, brown, black, and a wide range of related hues and colorations. Alternatively, the toothbrush body or portions of the toothbrush body can be pre-colorized to match either the initial sensing/reporting element color or the final sensing/reporting element color. These configurations provide a significant color change contrast and eliminate the ambiguity associated with the entire toothbrush changing color without any reference colors present.

The following examples are offered by way of illustration and not by way of limitation.

### EXPERIMENTAL

### Example 1

Brushing duration sensor using optical coating formulation: An optical coating formulation possessing a thermochromic sensing liquid formulation was prepared. An ultraviolet light sensitive resin was used as base coating composition (Nazdar 3227 UV mixing clear, Nazdar Corporation). 4% by weight dry powder thermochromic colorant (Keystone Corporation, Il, blue 29 degrees centigrade temperature transition) was added to the UV resin and mixed thoroughly to a uniform state (room temperature). Coating compositions were prepared in advance of use and stored at room temperature. Additional compositions were prepared with higher and lower temperature thermochromic agents including 25, 27, 31, 35 and 37 degrees centigrade).

### Example 2

UV/thermochromic optical indicating brushing duration sensor: The back surface of a polypropylene tooth brush head was surface roughened using a 300 grit sand paper. The surface was roughened to ensure good adhesion between the UV/thermochromic coating and the polypropylene surface. Alternatively, surfaces can be treated using a corona discharge to enhance the hydrophilic property of the surface. The UV/thermochromic liquid formulation was applied directly to the treated brush head surface. 20 micro liters were applied and allowed to spread out directly into a circular pattern.

The wetting angle of the fluid was such that the edges of the applied region thinned to 0.05 inches. The UV/thermochromic solution was rapidly cured using a UV lamp system (1200 watts per square inch, medium pressure lamp). The applied solution was initially quickly exposed to the UV lamp at a distance of 10 inches for 2 seconds. The curing process was prolonged to ensure minimal over heating and reduced potential to photo-bleach the color components. The applied solution was subsequently exposed more intensely at a distance of 6 inches from the lamp source for 4 seconds. The coating became firmly cured by touch to a hardened solid state.

### Example 3

Printed message brushing duration sensor using over-coated with UV/thermochromic window: A message was screen printed on the back-side of a toothbrush head using a stationary UV ink. The printed message was rapidly cure printed message using intense UV light source. The message was over-coated using a UV/thermochromic liquid formulation (above). The over-coat UV/thermochromic liquid was rapidly cured using the same light source as was used for curing the stationary UV ink.

Messages can be either printed using a silk screen method or an alternative method such as pad printing. The ink was applied in a thickness sufficient to clearly delineate the message of interest and provide for suitable adhesion characteristics to the toothbrush surface. For UV treatment and curing of UV stationary inks, a medium pressure mercury vapor UV lamp system (1200 watts/inch squared) was employed. Printed samples were exposed at full intensity for 3 seconds at a distance of 6 inches from the bulb. Care was taken not to over expose the ink formulation or over heat any component of the toothbrush. Message printed toothbrush samples could be further processed immediately or stored for further processing late a later stage.

A UV/thermochromic optical coating was placed over the message by applying the coating from a low pressure liquid applicator. A metered amount of UV/thermochromic solution was applied such that the coating self-leveled on the brush surface resulting in a thickness of 0.05 inch at the central height. The wetting angle of the fluid was such that the edges of the applied region thinned to 0.05 inches. The UV/thermochromic solution was rapidly cured using the same UV lamp system described above. The applied solution was initially quickly exposed to the UV lamp at a distance of 10 inches for 2 seconds. The curing process was prolonged to ensure minimal over heating and reduced potential to photo-bleach the color components. The applied solution was subsequently exposed more intensely at a distance of 6 inches from the lamp source for 4 seconds. The coating became firmly cured by touch to a hardened solid state.

The advantages of using optical UV coatings containing the thermochromic agent to obscure a message includes the ease of changing the message from time to time as desired and the use of low cost materials that involve only a limited amount for production of a workable toothbrush product. Materials and components can be specified and formulated to be in compliance with the Food and Drug Administration's criteria for use in oral contact applications.

### Example 4

Simplified hot melt formulation for duration sensor applications: A simplified hot melt composition was formulated in order to comprise a brush timing duration indicator. A low melt temperature polyethylene resin (melt index of 4000 at 300 degrees F.) was utilized as the base material. 300 gram resin was heated to 350 degrees F. in a convention oven. 8% by weight master-batched thermochromic colorant (Matsui International, Inc., Gardena Calif. Type 27 degrees C. color to colorless pigment, polyethylene master batch) was added to the molten resin and mixed thoroughly. 0.1% to 1.0% by weight of a compatible fluorescent dye pigment was added to create a bright color to color change (Dayglo Company). The fluorescent dye pigment was mixed uniformly into the molten resin.

After the indicating hot melt composition was completely blended, hot melt sticks were cast by pouring the mixture into rod-shaped silicon rubber molds. The molds formed ⅜ inch in diameter rounded rod shapes 6 inches in length. The cast rods were immediately ready for use once they had been cooled to room temperature and removed from the mold.

### Example 5

Strengthened adhesive hot melt formulation for duration sensor applications: The simplified hot melt composition described above was modified with a tackifying agent. A low melt temperature polyethylene resin (melt index of 4000 at 300 degrees F.) was utilized as the base material. 300 gram resin was heated to 350 degrees F. in a convention oven. 8% by weight master-batched thermochromic colorant (Matsui International, Inc., Gardena Calif. Type 27 degrees C. color to colorless pigment, polyethylene master batch) was added to the molten resin and mixed thoroughly. 01% to 1.0% by weight of a compatible fluorescent dye pigment was added to create a bright color to color change (Dayglo Company). The fluorescent dye pigment was mixed uniformly into the molten resin. 15% by weight tackifying agent (Arizona Chemical Comp. AZ) was added to the simplified formulation. The tackifying agent was blended to uniformity with the other composition components.

After the indicating hot melt composition was completely blended, hot melt sticks were cast by pouring the mixture into rod-shaped silicon rubber molds. The molds formed ⅜ inch in diameter rounded rod shapes 6 inches in length. The cast rods were immediately ready for use once they had been cooled to room temperature and removed from the mold.

### Example 6

Hardening hot melt formulations: Hot melt formulations can be hardened for improved integrity during use. In particular, it is important to minimize scratches and abrasions to the sensor surface during usage. Hardening polyolefin waxes can be added to improve the overall strength of a formulated sensor element. Hot melt formulations made similar to Example 5 above “Strengthened adhesive hot melt formulation for duration sensor applications”. Were strengthened using 15% hardened polyolefin wax. The wax was added during the heated stage and all compositions were completely melted and mixed thoroughly.

### Example 7

Bulk production preparation of sensing/reporting hot melt compositions: Hot melt compositions were formulated similar to described above. Bulk preparations were prepared with 8% by weight, master-batched thermochromic pigments (Matsui International, Gardena, Calif.). Resins were prepared with pre-formulated hot melt compositions (industrial adhesives ink, OR) or pure polyethylene resins (polyethylene polymer, 4000 centipoises at 350 degrees F.). Bulk formulations were prepared by initially pre-melting the hot melt resin 350 degrees F. for 1 hour or until liquefied. The hot melt composition was maintained in a clear liquid state prior to adding the master batched thermochromic pigment (polyethylene resin based). The thermochromic master-batched pigment was added in pellet form over the surface of the molten hot-melt resin. The entire composition was maintained at 350 degrees F. for 15 minutes or until the pigment was completely molten. Once the pigment was completely molten, the composition was stirred until the formulation was completely mixed. Complete mixing was achieved once all of the master-batched pigment resin was homogeneously dispersed.

Pre-sized and weighed ingots were prepared from the molten composition. Typically 1 lb ingots were prepared by pouring the molten composition into cylindrical nonstick tubing approximately 3 inches in diameter and 1 foot in length. The molten composition was easily weighed by placing the nonstick tubing vertically on a nonstick platform and sealing the two components together. The hot melt formulation was directly poured into the open top of the cylinder until the desired weight was achieved. Cylindrical canisters were cooled to room temperature by submersion in a water bath. For use, the ingots were removed from the cylinders by lightly tapping them and sliding them out. The preformed ingots could be used directly in standard hot melt automation equipment (e.g. ITW Dynatec Corporation)

### Example 8

Hot melt duration sensor in circular indent format: A polypropylene toothbrush head was prepared by machine milling a 0.25 inch in diameter hole 0.05 inch deep. The impression was milled in the brush head to contain and be filled by the hot melt formulation. The hot melt indicating materials cast into rods were melted in a low temperature hot melt gun. The indicating hot melt formulation was extruded into the machined indent.

Approximately 50 milligrams hot melt indicating formulation was applied to fill the indent. The indent was filled to completion with the surface level and flush with the upper level of the toothbrush surface. The molten hot melt indicating formulation was flattened using a flattened anvil coated with a silicone rubber coating. The coating prevented any sticking. The flattened anvil surface helped to level the hot melt formulation and accelerate cooling to a solidified state. The indicating brush was ready for use immediately after cooling the sensor to room temperature.

### Example 9

Automated duration sensor production process: An automated process for sensor/reporting toothbrush duration sensors was developed. Injection molded toothbrushes with a circular indented recess as described above were used in combination with production processed thermochromic sensing/reporting compositions also described above. The sensing/reporting compositions were utilized in automated hot melt production processing equipment (ITW Dynatec Corp.) The system was set up as follows: The hot melt reservoir was maintained at 350 degrees F. for melting the hot melt composition. The reservoir was filled with between 5-10 lbs of the hot melt formulation. A heater connector hose was maintained at 350 degrees F. and under low pneumatic pressure. High precision applicator nozzle was maintained at 350 degrees F. The applicator nozzle on an X, Y, Z positioning stage. Each system component was controlled using PLC controllers and algorithms. Pressures, nozzle sizes and process parameters were controlled such that the exact volume equivalent of hot melt composition could be delivered to the indented recess on a toothbrush. Sequential toothbrush fixtures and holders were adapted to a step and repeat linear transition stage (also computer controlled). The step and repeat functions as well as a hot well melt applicator functions were programmed such that an accurate volume of hot melt composition could be applied to a toothbrush every 2.5 seconds. The system design is capable of applying the hot melt composition to two toothbrushes simultaneously. The total system throughput was capable of applying continuous production runs with high throughput.

### Example 10

Hot melt duration sensor in patterned format: A hot melt duration sensor format was produced as described above. The indent was machined as a word pattern “DONE”. The wording was engraved in the toothbrush head using a high-speed engraver. The hot melt sensing formulation was prepared according to Example 5 above “Strengthened adhesive hot melt formulation for duration sensor application”. The color of the cold sensing formulation was matched with the color of the polypropylene brush handle. During use, the word “done” appeared during brushing due to the color change as brushing progressed.

### Example 11

Indicating color change dental chewing gum: A thermochromic temperature indicating chewing gum was prepared to monitor oral cavity temperatures. The chewing gum was capable of indicating normal body temperatures and/or fever elevated body temperatures greater than 99 degrees F. Chewing gum base was blended with a sugar substitute (0.5% by weight), corn starch (10% by weight), and a thermochromic pigment (2% by weight, temperature transition set at 99 degrees F., blue to clear leuco dye) and a colorfast red food dye (Red 28, FDC lake pigment). The composition was blended under molten conditions at 250 degrees F. for 20 minutes and then processed into 0.5 inch wide 1.0 inch long and 0.2 inch thick tiles.

The formulated gum was a deep purple color at room temperature. Upon chewing and below 99 degrees F., the gum remained light purple. When oral temperatures at or above 99 degrees F. were monitored, chewing resulted in the gum transitioning from a purple color to a red color. The chewing gum remained red so long as chewing continued and until the gum was expelled. The chewing gum could accurately be used to monitor fever level temperatures at or above 99 degrees F.

### Example 12

Toothbrush life-time sensor using hot melt formulation: A medium temperature hot melt formulation can be utilized as an irreversible color change indicator for determining the useful life-time of a used toothbrush. The sensor formulation can comprise a colorless to color change or a color to color change such that utilization of a formulated sensor causes an irreversible color change over a prolonged and specified period of time reflecting the useful life-time of the toothbrush. The mechanical shear and contact effect of brushing during which teeth and gums come in contact with the sensor results in a structural impact on the surface of the sensor. The structural impact result in structural shear force change in the sensor composition that disrupts encapsulated leuco-dyes resulting in a distinct color change. The degree of structural shear force over time can be directly related to the total usage of the toothbrush. The composition can be formulated accordingly to accommodate a manufacturer's suggested usage duration.

### Example 13

Pressure sensitive brushing duration polyethylene films for pressure sensitive adhesive attachment: Polyethylene resin (General Polymers, division of Ashland Chemicals: PE HUN2051 NT) was co-extruded with 7% by weight master batch polyethylene thermochromic colorant (Matsui Corporation, CA: type 27° C. color to colorless or color to color). Colors utilized to make sheets included green to colorless, magenta to colorless, blue to colorless, orange to colorless, yellow to colorless, green to yellow, purple to red, purple to blue, green to orange and orange to yellow. Extrusion was performed with a 1 inch extruder using a mixing screw and 3 heated zones (Wayne Machines, Inc.: zone 1 at 390° F., zone 2 at 350° F., and zone 3 at 325° F.). Extruded rods were formed and then stored for later use.

Films were compression molded using a 125 ton heated platen hydraulic press (Wabash Corporation: 6 opening 28 inch by 28 inch platen size). Initially, the extruded plastic rods (½ inch in diameter, 8 inch in length) were preheated and softened at 275° F. for 10 minutes. The 400 gram preheated rods were placed between preheated Teflon coated aluminum sheets (⅛ inch thick, 28 inch by 28 inch square, preheated to 300° F.). The sheet/rod sandwich was placed in the pre-heated platen press (preheated to 300° F.). The rods were pressed to a sheet thickness of 0.05 inch.

Pressure sensitive adhesive in a double stick format (3M Corporation, 0.005 inch thick, general purpose) was applied to one side of a pre-formed polyethylene sheet. Tabs were die cut from the pressure sensitive sheet stock using a steel rule die (0.25 inch wide, 0.5 inch long with a 0.125 inch rounded radius at each end). The die cut pressure sensitive sensor tabs could be directly adhered to the back side of toothbrush heads. The pressure sensitive adhesive was firmly seated using moderate pressures for application.

### Example 14

Pressure sensitive film applied as a brushing duration sensor: The die cut sensor tab/label prepared as described above, was affixed on nested brush directly on the back-side of the toothbrush head. The tab was firmly adhered using a pressure sensitive adhesive laminated on the sensor tab/label. The toothbrush was immediately ready for use. In similar cases the sensor tab could be adhered using hot melt adhesives, heat laminated using a high-pressure heat laminator, or sonically welded using a sonic welding apparatus.

### Example 15

Brushing duration sensor showing time progression: The duration indicator was prepared with a depth gradient well from 0.005 inch to 0.050 inch. The well was machined into a polypropylene toothbrush handle. The total well length was 2 centimeters and was machined on the back side of the toothbrush head. The sensing composition was prepared according to Example 5 above “Strengthened adhesive hot melt formulation for duration sensor applications”. The thinner region changes color first during brushing to indicate that brushing is partially complete. As the brushing time comes to completion, the thicker portion of the indicator turns color. A complete color change along the length of the indicator indicated that brushing has been accomplished for the expected duration. The progression of brushing timing was easy to follow as the color changed along the axis of the gradient well.

### Example 16

Toothbrush use and expiration sensor: A toothbrush use and expiration sensor was generated using a hot melt composition including an encapsulated leuco dye. The leuco dye was combined with a medium temperature polyethylene resin and a hot melt adhesive composition. The leuco dye was added at 3% by weight powder (Color Change Corporation). The leuco dye utilized had a stated temperature transition of 22° C. The polypropylene resin was added at 30% by weight (medium density polypropylene from Ashland Chemicals).

### Example 17

Toothbrush duration indicator with embossed reflective and holographic patterns: The hot melt surface can be embossed, textured, or finished with a holographic pattern. The embossing or patterning process can occur immediately upon pressing a deposited molten hot melt sensing material. The cooling and flattening surface intended for pressing the molten composition can be patterned with the reverse image intended to be impressed into the surface.

Other oral care product examples include, but are not limited to: a brushing duration sensor that uses a liquid crystal gel label format; dental floss use-based color change as an indicator composition for proper usage and timing; plural indicators for brushing duration and for brush life-time; a gum stimulator duration sensor for indicating appropriate usage timing; a toothbrush duration indicator with a green/go color change to a red/stop brushing sensor timer; fluorescent/thermochromic colorants for brushing duration sensor applications; metallic flake/thermochromic colorants for brushing duration sensor applications and related examples.

Additional product examples may include, but are not limited to: warning indicating dots on consumer products; tamper evident indicators indicating that a product has been violated prior to purchase or intended use; automotive products where use or over-use should be indicated; household cleaning products where it is important to indicate levels, strength, temperature-of-use or the like; food products where it is important to indicate expiration, freshness; intended storage temperatures, tamper or the like; medicinal products where it is important to indicate sequence of use, storage temperature, storage conditions or the like, toys where it is important to indicate intended play patterns, proper usage, replacement time of a feature, safety features, new toy embodiments or features; usage and “Try Me” features on cosmetic products; novel packaging elements where, for example, hot melt compositions can be used not only for glue, but for interactive purposes such as “Try Me” features, interactive features, attractive color change features, and the like; and household mending and gluing products such hot melt guns, glue guns and hot melt resins where it is important that the applied resin can have a new color change component and sensing/reporting characteristics.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An intrinsic visual optical change sensor configured for use with an oral product.

2. The sensor according to claim 1, wherein said intrinsic visual optical change sensor undergoes a chromic change in response to a stimulus.

3. The sensor according to claim 2, wherein said stimulus is a duration stimulus.

4. The sensor according to claim 3, wherein said duration stimulus comprises exposure to a temperature in excess of a predetermined temperature for a predetermined amount of time.

5. The sensor according to claim 2, wherein said chromic change is a change from a first color to a second color.

6. The sensor according to claim 2, wherein said chromic change is a change from a hued to a hueless state.

7. The sensor according to claim 1, wherein said oral product is a toothbrush.

8. The sensor according to claim 1, wherein said oral product is a dental floss.

9. The sensor according to claim 1, wherein said oral product is a gum.

10. The sensor according to claim 1, wherein said oral product is a tongue cleaner.

11. The sensor according to claim 1, wherein said oral product is a gum stimulator.

12. The sensor according to claim 1, wherein said change is reversible.

13. The sensor according to claim 1, wherein said change is irreversible.

14. The sensor according to claim 1, wherein said sensor is adhered to a portion of an oral care product that is configured for placement in an oral cavity.

15-25. (canceled)

26. A hot melt composition comprising an intrinsic detection element.

27. The hot melt composition according to claim 26, wherein said intrinsic detection element provides an intrinsic optical signal in response to a stimulus.

28-51. (canceled)

52. An oral care product comprising a sensor adhered to a portion of said product that is configured for placement in an oral cavity, wherein said sensor comprises an applied hot melt composition comprising an intrinsic detection element.

53-62. (canceled)

63. A method for producing an oral care product, said method comprising:
preparing a hot melt composition comprising an intrinsic detection element; and applying said hot melt composition to a location on an oral care product.

64-148. (canceled)
